(19) United States
(12) Patent Application Publication
(10) Pub. No.: US 2026/0294867 A1
(43) Pub. Date: Oct. 1, 2026

(54) METHODS FOR WEIGHT LOSS AND/OR REDUCING WEIGHT GAIN

(71) Applicant: The Medical College of Wisconsin, Inc., Milwaukee, WI (US)

(72) Inventors: John Kirby, Shorewood, WI (US); Matthew Hadiono, Milwaukee, WI (US)

(21) Appl. No.: 19/478,084

(22) PCT Filed: May 30, 2024
(86) PCT No.: PCT/US2024/031710

§ 371 Date: Oct. 23, 2025

Related U.S. Application Data
(60) Provisional application No. 63/505,036, filed on May 30, 2023.

(51) Int. Cl. A61K 31/4015; A61K 9/00; A61K 31/70; A61K 31/7004; A61K 31/7042; A61K 31/7048; A61K 31/7056; A61K 38/26; A61P 3/04

(52) U.S. Cl. CPC A61K 31/4015; A61K 9/0019; A61K 31/70; A61K 31/7004; A61K 31/7042; A61K 31/7048; A61K 31/7056; A61K 38/26; A61P 3/04

(57) ABSTRACT

Disclosed herein are methods for weight loss and/or for reducing weight gain. The methods can include administering to a subject reutericyclin and a GLP-1 receptor agonist. Additional methods for weight loss and/or reducing weight gain include administering to a subject reutericyclin and a SGLT-2 inhibitor.

### STATEMENT REGARDING FEDERALLY SPONSORED RESEARCH OR DEVELOPMENT

None.

### BACKGROUND

Excessive weight can be associated with one or more medical conditions and/or can lead to numerous medical conditions.

### SUMMARY

Disclosed herein are methods for weight loss and/or for reducing weight gain.

In various aspects, a method for weight loss in a subject is disclosed. The method can include administering a therapeutically effective amount of reutericyclin and a glucagon-like-protein-1 (GLP-1) receptor agonist to the subject.

In various aspects, a method for weight loss in a subject is disclosed. The method can include administering a therapeutically effective amount of reutericyclin and a sodium glucose cotransporter 2 (SGLT-2) inhibitor to the subject.

### BRIEF DESCRIPTION OF THE FIGURES

FIG. 1 is a schematic depiction of the experimental approach for Example 1.

FIGS. 2A and 2B depict two graphs illustrating the weight in grams of the mice in Example 1 over the three week test period. FIG. 2A depicts the weight of the four cohorts of mice over the 21 days of the test (with day zero starting at 14 weeks). FIG. 2B depicts the day 21 (17 weeks of age) body mass of the mice from each group. Each dot on the bar graph represents an individual mouse. Error bars show standard error.

FIGS. 3A and 3B depict two graphs illustrating the change in body mass in grams of the mice in Example 1 over the three week test period. FIG. 3A depicts the change in mass (g) over the 21 days of the test. FIG. 3B depicts the change in body mass at day 21. Each dot on the bar graph represents an individual mouse. Error bars show standard error.

FIGS. 4A and 4B depict two graphs depicting the eating behavior and fasted blood glucose levels of the mice from Example 1. FIG. 4A depicts the calories ingested per day by each of the mice in each group, while FIG. 4B depicts fasted blood glucose levels in each of the mice of each group. Each dot on the bar graph represents an individual mouse. Error bars show standard error.

FIG. 5 is a schematic depiction of the experimental approach for Example 2.

FIGS. 6A and 6B depict two graphs illustrating the weight change of the mice in Example 2. FIG. 6A depicts the change in body mass (grams) over 26 days. FIG. 6B depicts the change in body mass on day 26, with day 1 being the start of the experiment.

FIG. 7 is a bar graph depicting the change in weight after withdrawal of the semaglutide at day 46 of the experiment described in Example 2.

FIG. 8 is a bar graph depicting the feeding efficiency of the mice as described in Example 2.

FIG. 9 is a bar graph depicting the fasted blood glucose level at day 27 as described in Example 2

### DETAILED DESCRIPTION

### Overview

Excessive weight can be associated with one or more medical conditions and/or can lead to numerous medical conditions. Glucagon-like Peptide-1 (GLP-1) receptor agonists are a class of medications used to treat type 2 diabetes and obesity, in certain cases. However, GLP-1 receptor agonists have side effects when taken long term. When patients stop taking the GLP-1 receptor agonists, most of the benefits, especially weight loss, appear to be lost. For instance, in certain scenarios, patients regain all the weight loss attained from taking the GLP-1 receptor agonist and can have additional pathologies once they cease taking the GLP-1 receptor agonist.

Sodium glucose cotransporter 2 (SGLT-2) inhibitors are another class of compounds used to treat type 2 diabetes and can also produce weight loss. However, SGLT-2 inhibitors also may cause side effects and/or may not be an ideal medication for weight loss treatment.

There is a need for additional methods and compositions for treating weight loss and/or reducing weight gain.

Reutericyclin is a small molecular weight antimicrobial produced by some strains of Lactobacillus reuteri. In various aspects, the methods disclosed herein relate to a combination therapy including the administration of reutericyclin and a GLP-1 receptor agonist. In one or more aspects, the methods disclosed herein relate to a combination therapy including the administration of reutericyclin and a SGLT-2 inhibitor. In the same or alternative aspects, the methods disclosed herein relate to a combination therapy including the administration of reutericyclin and a GLP-1 receptor agonist and/or a SGLT-2 inhibitor.

In various aspects, the methods disclosed herein that utilize combination therapies (e.g., reutericyclin and a GLP-1 receptor agonist) can allow for the use of a reduced dose of a GLP-1 receptor agonist without sacrificing the weight loss results attained on a larger dose of the GLP-1 receptor agonist. In such aspects, the reduced dosage of the GLP-1 receptor agonist may aid in preventing and/or reducing the unwanted side effects experienced on higher doses of the GLP-1 receptor agonist. In certain aspects, the combination therapies can also include a SGLT-2 inhibitor in place of, or together with, the GLP-1 receptor agonist.

In various aspects, the methods disclosed herein can allow for a combination therapy and/or co-administration of reutericyclin and a GLP-1 receptor agonist followed by administration of reutericyclin in the absence of the GLP-1 receptor agonist, while still retaining the benefits, e.g., reduced weight gain and/or weight loss, of the combination therapy. In certain aspects, the combination therapies can also include a SGLT-2 inhibitor in place of, or together with, the GLP-1 receptor agonist.

### Definitions and Terminology

The disclosed methods for weight loss and/or for reducing weight gain, may be further described using definitions and terminology as follows. The definitions and terminology used herein are for the purpose of describing particular aspects only and are not intended to be limiting.

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “subject” may be used interchangeably with the terms “individual” and “patient” and includes human and non-human subjects. In some embodiments, subjects may be any mammal.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The phrase “reducing weight gain” refers to any indicia of success in the prevention or reduction of weight gain in a patient. The prevention or reduction of weight gain can be measured based on objective parameters, such as the results of a physical examination. In certain aspects, reducing weight gain in a patient can be determined by comparing the weight of the patient taken at two time points, where the time points are separated by at least one week.

The phrase “weight loss” refers to a loss or reduction in the weight of a patient. Such a loss or reduction in the weight of a patient can be measured based on objective parameters, such as the results of a physical examination. In certain aspects, weight loss can be the loss or reduction in the weight of a patient as determined by comparing the weight of the patient taken at two time points, where the time points are separated by at least one week.

A non-limiting example of a treatment for a subject includes, but is not limited to, administering a therapeutically effective amount of reutericyclin and a glucagon-like-protein-1 (GLP-1) receptor agonist to the subject. Another non-limiting example of a treatment for a subject includes, but is not limited to, administering a therapeutically effective amount of reutericyclin and a sodium glucose cotransporter 2 (SGLT-2) inhibitor to the subject.

A non-limiting example of a preventative treatment for a subject includes, but is not limited to, administering a therapeutically effective amount of reutericyclin and a glucagon-like-protein-1 (GLP-1) receptor agonist to the subject. Another non-limiting example of a preventative treatment for a subject includes, but is not limited to, administering a therapeutically effective amount of reutericyclin and a sodium glucose cotransporter 2 (SGLT-2) inhibitor to the subject.

As used herein, “therapeutically effective amount” refers to an amount of a therapeutic agent effective to cause weight loss and/or reduce weight gain in a subject.

### Reutericyclin, GLP-1 Receptor Agonists, and SGLT-2 Inhibitors

In various aspects, reutericyclin refers to a highly hydrophobic, charged tetramic acid molecule produced by and isolated from particular strains of Lactobacillus reuterii. In certain aspects, the strain can be, but is not limited to, Lactobacillus reuterii LTH2584. Referring to the main tautomeric form in solution, reutericyclin is (5R)-1-(2-decenoyl)-2-hydroxy-3-acetyl-5-isobutyl-42-pyrroline-4-one, a N-acylated tetramic acid. N-alkyl reutericyclin analogs have been shown to retain activity against Gram-positive pathogens and other functional properties of reutericyclin. See, e.g., Yendapally et al., J. Med. Chem. 51, 1487-1491 (2008); Hurdle et al., Antimicrob. Agents Chemother. 53, 4028-4031 (2009). In various aspects, the reutericyclin is purified, e.g., from a cell lysate prior to administration. In alternative aspects, the reutericylin is administered as part of a reutericyclin-producing Lactobacillus reuterii lysate or reutericyclin-producing Lactobacillus reuterii bacteria. It should be understood that the methods disclosed herein contemplate the use of reutericyclin and analogues of reutericyclin.

In various aspects, GLP-1 receptor agonists refer to a class of compounds that bind to the GLP-1 receptor. In various aspects, the GLP-receptor agonists can include glucagon-like peptide-1 (GLP-1), GLP-1 variants, and/or GLP-1 peptide mimetics.

In various aspects, the GLP-1 variants can be conservatively modified variants. One of skill will recognize that individual substitutions to a peptide, polypeptide, or protein sequence which alters a single amino acid is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

In various aspects, GLP-1 peptide mimetics refer to a molecule, including peptides, modified peptides, or any other molecule that biologically mimics the activity of the GLP-1 peptide.

A non-limiting list of example GLP-1 receptor agonists includes: semaglutide, dulaglutide, exenatide, liraglutide, and lixisenatide, which are all commercially available. In certain aspects, the GLP-1 receptor agonist can comprise one or more of semaglutide, dulaglutide, exenatide, liraglutide, or lixisenatide. In one or more aspects, the GLP-1 receptor agonist is one or more of semaglutide, dulaglutide, exenatide, liraglutide, or lixisenatide.

In one or more aspects, the GLP-1 receptor agonists disclosed herein may also be part of a combination therapeutic, separate from reutericyclin. For instance, the GLP-1 receptor agonists disclosed herein are contemplated to also include therapeutics that include a GLP-1 receptor agonist and another therapeutic agent. In various aspects, the other therapeutic agent may be a GIP (glucose-dependent insulinotropic polypeptide) receptor agonist. One non-limiting example of a GLP-1 receptor agonist that is part of a combination therapeutic is tirzepatide, which is a commercially available therapeutic that includes semaglutide and a GIP receptor agonist. In this one non-limiting example, tirzepatide is a single molecule. However, also contemplated herein are combination therapeutics that include a GLP-1 receptor agonists as part of combination therapeutics, where each of the therapeutics are separate molecules.

In various aspects, the SLGT-2 inhibitors refer to a class of compounds that can block the low-affinity, high-capacity SGLT2 protein, which can result in lowering of plasma glucose concentrations. See e.g., Madaan et al., Sodium glucose CoTransporter 2 (SGLT2) inhibitors: Current status and future perspective, Eur J Pharm Sci. 2016 Oct. 10; 93:244-52.

A non-limiting list of SLGT-2 inhibitors includes: bexagliflozin, canagliflozin, dapagliflozin, empagliflozin, ertugliflozin, ipragliflozin, luseogliflozin, remogliflozin, sergliflozin, sotagliflozin, or tofagliflozin, which are commercially available.

### Methods

In various aspects, methods for weight loss and/or for reducing weight gain in a subject are disclosed.

In various aspects, the subject is obese and/or is diagnosed with diabetes. In certain aspects, the subject is diagnosed with type 2 diabetes. Obesity can be measured based on objective parameters, such as the results of a physical examination and may include the use of body mass index.

In various aspects, the methods can include administering a therapeutically effective amount of reutericyclin and a GLP-1 receptor agonist to a subject. In various aspects, the GLP-1 receptor agonist and reutericyclin can include any or all of the parameters described above with respect to these compounds.

The reutericyclin can be administered in any amount that is therapeutically effective. In various aspects, the reutericyclin can be administered in an amount of from 10 μg/kg of body weight to 500 μg/kg of body weight; or 50 μg/kg of body weight to 300 μg/kg of body weight; or 500 μg/kg of body weight or less 300 μg/kg body weight or less; or about 100 μg/kg of body weight.

The GLP-1 receptor agonist can be administered in any amount that is therapeutically effective. As discussed herein, when the GLP-1 receptor agonist is administered along with reutericyclin, lower amounts of GLP-1 receptor agonist can be utilized, while retaining the benefits attained on higher doses of the GLP-1 receptor agonist. For instance, when reutericyclin is administered along with a GLP-1 receptor agonist at a low dose, it was unexpected to discover that the weight loss results are similar to those obtained when a subject is administered a dose of GLP-1 receptor agonist that is at least ten times higher than the low dose and in the absence of reutericyclin.

In certain aspects, the GLP receptor agonist can be administered in an amount of 1 nanomole/kg of body weight or less, 0.1 nanomoles/kg of body weight or less, or 0.01 nanomoles/kg of body weight or less, or 0.001 nanomoles/kg of body weight or less; or a range of from 0.001 nanomoles/kg of body weight to 1 nanomole/kg of body weight.

In various aspects, the reutericyclin and GLP-1 receptor agonist can be administered together or separately. As used herein, administering together refers to administering two therapeutic agents either at substantially the same time and/or via the same administration route. In various aspects, reutericyclin and the GLP-1 receptor agonist can be administered together in the same composition, e.g., pill, liquid, or the like, or as separate compositions. In one or more aspects, the reutericyclin and GLP-1 receptor agonist can be administered orally or via injection. In certain aspects, the reutericyclin can be administered orally. In the same or alternative aspects, the GLP-1 receptor agonist can be administered orally or via injection. In certain aspects, the GLP-1 receptor agonist can be administered via subcutaneous injection, intramuscular injection, or intraperitoneally.

In various aspects, the reutericyclin and/or the GLP-1 receptor agonist can be administered according to any suitable dosing schedule. In one or more aspects, the reutericyclin and/or the GLP-1 receptor agonist can be administered daily or weekly.

In certain aspects, the reutericyclin and GLP-1 receptor agonist can be administered to a subject for any suitable period of time. In certain aspects, the reutericyclin and GLP-1 receptor agonist can be administered to a subject for a defined duration of time, e.g., a period of one day to three years or more, or one day to 50 weeks, or one day to 26 weeks. In alternative aspects, the reutericyclin and GLP-1 receptor agonist can be administered to a subject for an undefined period of time.

In various aspects, the reutericyclin and GLP-1 receptor agonist can be administered to a subject for a first time period, such as one of the time periods discussed in the above paragraph, then for a second time period, reutericyclin may be administered to the subject in the absence of the GLP-1 receptor agonist. In such an aspect, the second time period can be any suitable length of time from days to years. In various aspects, during the second period of time the subject has gained less weight as compared to a subject receiving reutericyclin alone or GLP-1 receptor agonist alone during the first time period and receiving reutericyclin alone during the second time period.

In one or more aspect, methods described herein can include administering reutericyclin and a sodium glucose cotransporter 2 (SGLT-2) inhibitor to a subject. In various aspects, the SLGT-2 inhibitor and reutericyclin can include any or all of the parameters described above with respect to these compounds. In various aspects, the reutericyclin can be administered in any of the amounts discussed above.

In various aspects, the SGLT-2 inhibitor can be administered in any suitable amount. In certain aspects, the SGLT-2 inhibitor may be administered in an amount that is lower than would be administered to a subject in the absence of reutericyclin. That is, in various aspects, the administration of reutericyclin along with SGLT-2 may allow for a reduced dosing of the SGLT-2 inhibitor while retaining the benefit of the higher dosage.

In various aspects, the reutericyclin and SGLT-2 inhibitor can be administered together or separately. As used herein, administering together refers to administering two therapeutic agents either at substantially the same time and/or via the same administration route. In various aspects, reutericylcin and SGLT-2 inhibitor can be administered together in the same composition, e.g., pill, liquid, or the like, or as separate compositions. In one or more aspects, the reutericyclin and SGLT-2 inhibitor can be administered orally or via injection. In certain aspects, the reutericyclin can be administered orally. In the same or alternative aspects, the SGLT-2 inhibitor can be administered orally or via injection.

In various aspects, the reutericyclin and/or the SGLT-2 inhibitor can be administered according to any suitable dosing schedule. In one or more aspects, the reutericyclin and/or the SGLT-2 inhibitor can be administered daily or weekly.

In certain aspects, the reutericyclin and SGLT-2 inhibitor can be administered to a subject for any suitable period of time. In certain aspects, the reutericyclin and SGLT-2 inhibitor can be administered to a subject for a defined duration of time, e.g., a period of one day to three years or more, or one day to 50 weeks, or one day to 26 weeks. In alternative aspects, the reutericyclin and SGLT-2 inhibitor can be administered to a subject for an undefined period of time.

In various aspects, the reutericyclin and SGLT-2 inhibitor can be administered to a subject for a first time period, such as one of the time periods discussed in the above paragraph, then for a second time period, reutericyclin may be administered to the subject in the absence of the SGLT-2 inhibitor.

### EXAMPLES

The following Examples are illustrative and are not intended to limit the scope of the claimed subject matter.

### Example 1—Weight Loss in Combined Reutericyclin and Semaglutide Treatment

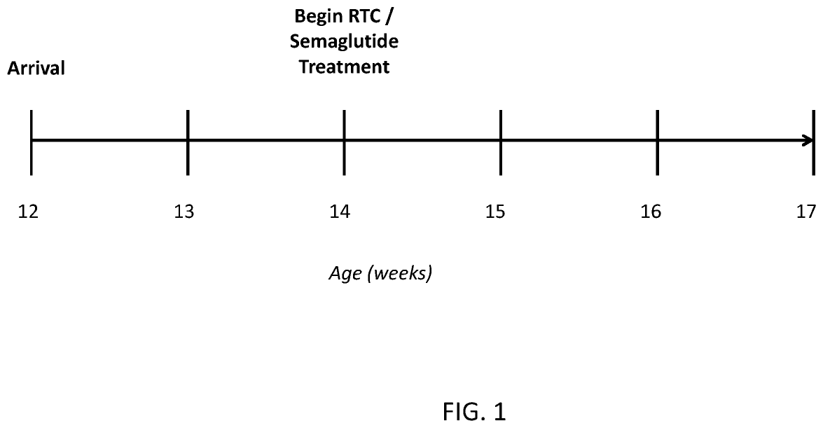

FIG. 1 depicts the experimental approach for this Example. Additional information for this approach is provided in Tables 1 and 2 below. C57BL/6J diet-induced obesity (DIO) male mice were obtained at 12 weeks of age and housed singly in the MCW animal facility (www.jax.org/jax-mice-and-services/strain-data-sheet-pages/phenotype-information-380050). Mice were maintained on a 60% high fat diet (HFD) (Research Diets D12492) throughout the experiment. Mice were acclimated for 2 weeks prior to treatment with either semaglutide, reutericyclin, both drugs, or neither drug.

TABLE 1
Experimental Approach - Mice

| | |
|---|---|
| Background | C57BL/6J DIO (380050) |
| Sex | Male |
| Housing | Single-caging upon arrival |
| Diet | 60% high-fat diet |

TABLE 2
Experimental Approach - experimental groups and number of mice

| | # of animals |
|---|---|
| HFD-PBS | 10 |
| HFD-RTC | 10 |
| HFD-RTC-SemaG | 10 |
| HFD-SemaG | 10 |

Reutericyclin was obtained from MedChemExpress (www.medchemexpress.com/reutericyclin.html). Semaglutide was obtained from BOC Sciences, catalog number BAT-010191.

Mice were weight-matched into 4 groups (N=10 per group) and given either reutericyclin via gavage to achieve a dosage of 100 μg/kg or injected intraperitoneally (IP) with semaglutide to administer 1 nmole/kg/day. All mice experienced both gavage and injection with either drug or vehicle. Vehicle for reutericyclin and semaglute are identical: 3.0% DMSO in phosphate buffered saline (PBS).

The four groups were: the control group (HFD-PBS), which received vehicle only; the HFD-RTC group, which were administered reutericyclin at a dose of 100 μg/kg of body weight in PBS; the HFD-RTC-SemaG group, which received reutericyclin at a dose of 100 μg/kg of body weight and 1 nanomole of semaglutide/kg of body weight in vehicle; and the HFD-SemaG group, which were administered 1 nanomole of semaglutide/kg of body weight in vehicle.

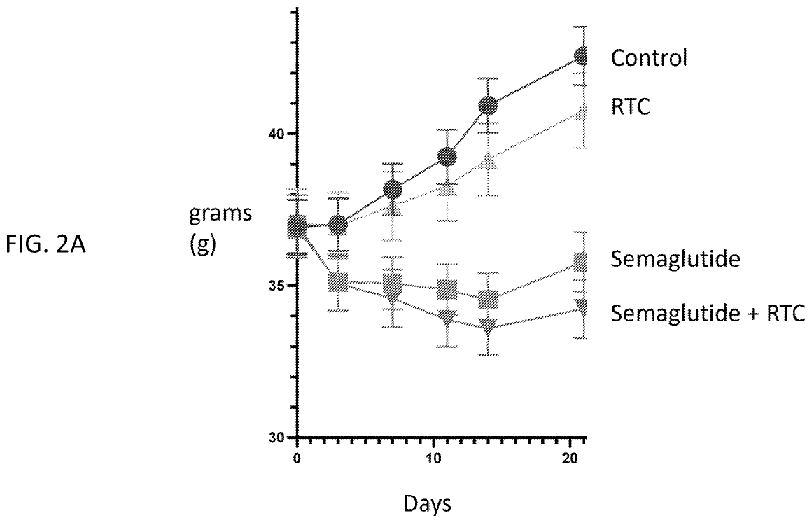

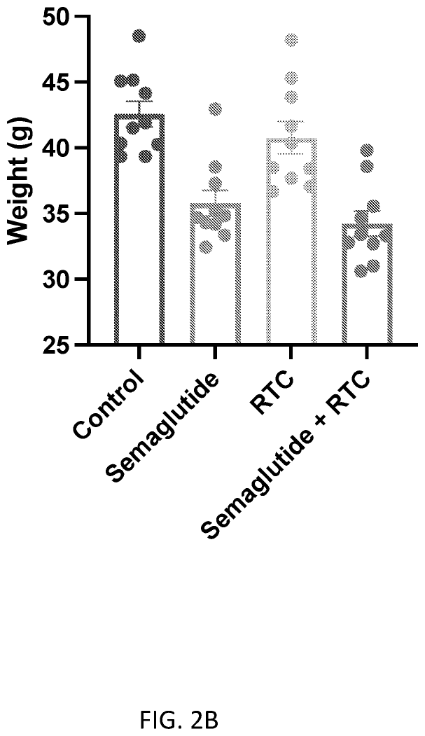

FIGS. 2A and 2B depict two graphs illustrating the absolute weight in grams of the mice in Example 1 over the three week test period. FIG. 2A depicts the weight of the four cohorts of mice over the 21 days of the test (with day zero starting at 14 weeks). The bar graph of FIG. 2B depicts the day 21 (17 weeks of age) body mass of the mice from each cohort. Each dot on the bar graph represents an individual mouse. Error bars show standard error. As can be seen in FIGS. 2A and 2B, the group receiving reutericyclin and semaglutide together exhibited greater weight loss as compared to every other cohort.

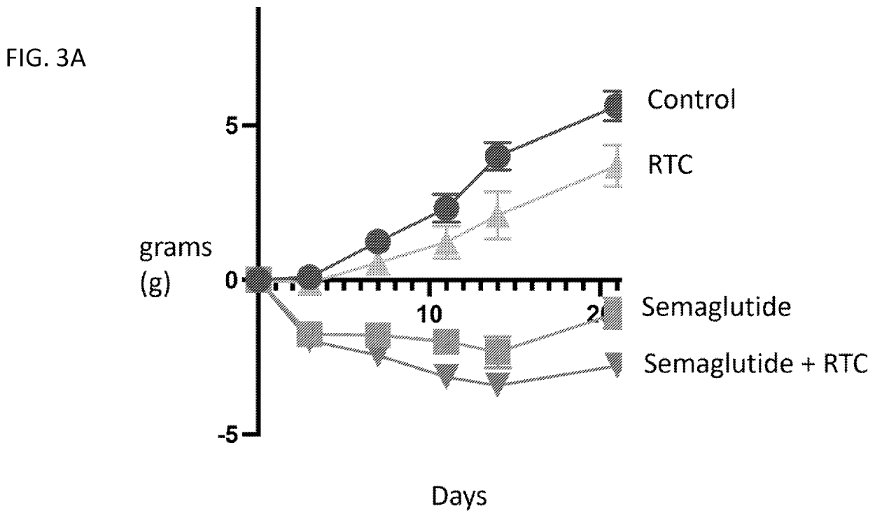

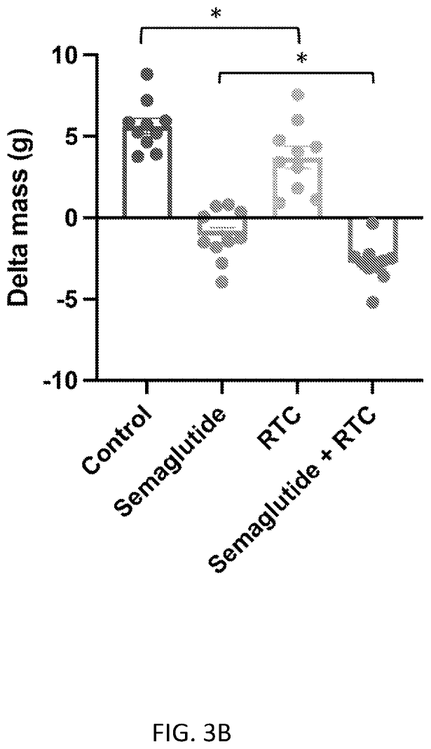

FIGS. 3A and 3B depict two graphs illustrating the change in body mass in grams of the mice in Example 1 over the three week test period. FIG. 3A depicts the change in mass (g) over the 21 days of the test. FIG. 3B depicts the change in body mass at day 21. Each dot on the bar graph represents an individual mouse. Error bars show standard error. As can be seen in FIGS. 3A and 3B the group receiving both reutericyclin and semaglutide together had the largest weight loss.

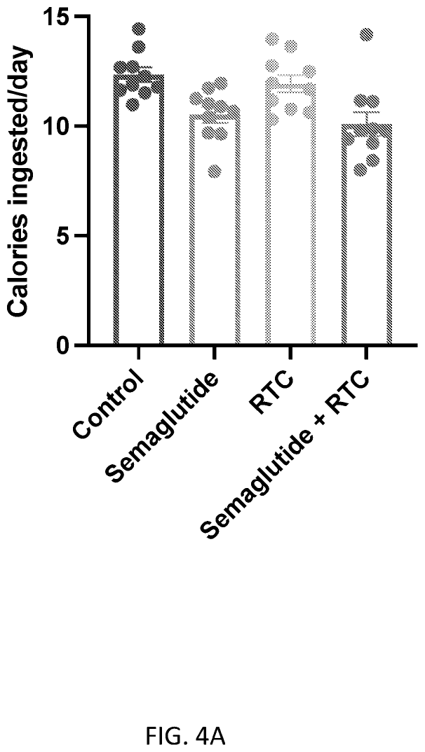

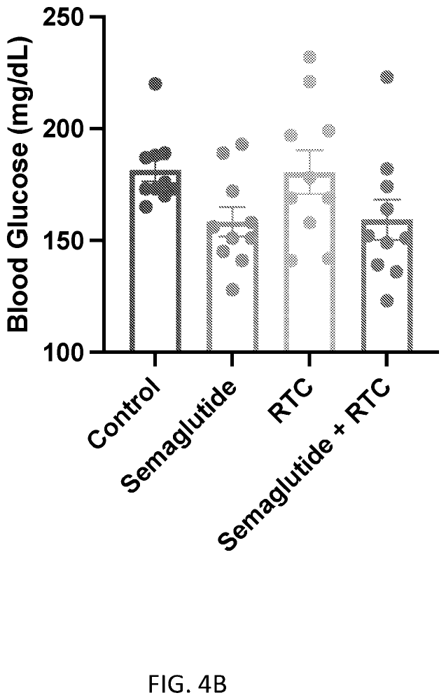

FIGS. 4A and 4B depict two graphs depicting the eating behavior (FIG. 4A) and blood glucose levels (FIG. 4B) of the mice from Example 1. FIG. 4A depicts the calories ingested per day by each of the mice in each group, while FIG. 4B depicts blood glucose levels in each of the mice of each group. Each dot on the bar graph represents an individual mouse. Error bars show standard error. 2-way ANOVA of these results in FIGS. 4A and 4B show that injection of semaglutide has a significant impact on eating and fasting blood glucose, and there was no noted effect of reutericyclin nor interaction of semaglutide and reutericyclin on eating and fasting blood glucose.

### Example 2—Combined Reutericyclin and Semaglutide Treatment for Three Weeks Followed by Reutericyclin Alone for Four Weeks

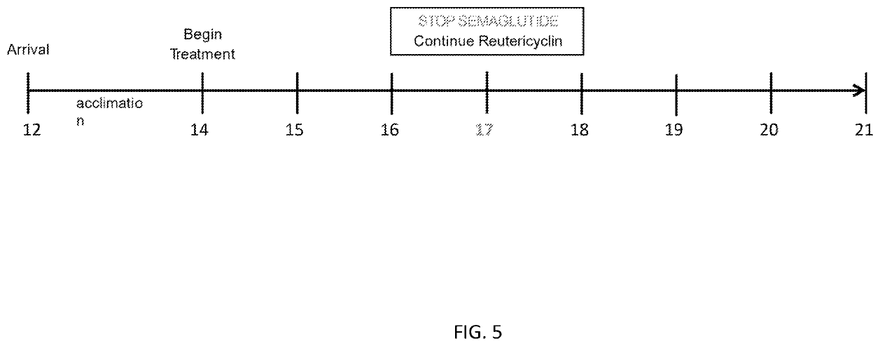

FIG. 5 depicts the experimental approach for this Example, and additional details can be found in Tables 3 and 4 below. C57BL/6J diet-induced obesity (DIO) male mice were obtained at 12 weeks of age and housed singly in the MCW animal facility (https://www.jax.org/jax-mice-and-services/strain-data-sheet-pages/phenotype-information-380050). Mice were maintained on a 60% high fat diet (Research Diets D12492) throughout the experiment. Mice were acclimated for 2 weeks prior to treatment with either semaglutide, reutericyclin (RTC), both drugs, or neither drug.

TABLE 3
Experimental Approach - Mice

| | |
|---|---|
| Background | C57BL/6J DIO (380050) |
| Sex | Male |
| Housing | Single-caging upon arrival |
| Diet | 60% high-fat diet |

TABLE 4
Experimental Approach - experimental groups and number of mice

| | # of animals |
|---|---|
| HFD-PBS | 10 |
| HFD-RTC | 10 |
| HFD-RTC-SemaG | 10 |
| HFD-SemaG | 10 |

Reutericyclin was obtained from MedChemExpress (https://www.medchemexpress.com/reutericyclin.html). Semaglutide was obtained from BOC Sciences, catalog number BAT-010191.

Mice were weight-matched into 4 groups (N=10 per group) and given either reutericyclin via gavage to achieve a dosage of 100 μg/kg or injected intraperitoneally (IP) with semaglutide to administer 1 nmole/kg/day. All mice experienced both gavage and injection with either drug or vehicle. Vehicle for reutericyclin and semaglute are identical: 3.0% DMSO in phosphate buffered saline (PBS).

The four groups of mice were as follows: Group 1 (black) (HFD-PBS) received no drug but experienced gavage and injection; Group 2 (red) (HFD-RTC) received reutericyclin via gavage and experienced injection with vehicle only; Group 3 (green) (HFD-SemaG) received no drug via gavage and experienced injection with semaglutide; and Group 4 (purple) (HFD-RTC-SemaG) received reutericyclin via gavage and experienced injection with semaglutide.

Treatment began at 14 weeks of age (timeline on arrow in FIG. 5) and continued for 26 days. Semaglutide treatment stopped for mice in groups 3 and 4 at day 26 (between 17 and 18 weeks of age) and measurements continued until day 46 of the experiment (approximately 21 weeks of age).

Mice were weighed daily to assess changes in body mass. Food hoppers were weighed daily to assess the amount of food consumed. Feeding efficiency is calculated by measuring the amount of weight gained by each mouse per gram of food consumed over a period of 5 days. Feeding efficiency shown here was calculated over days 21-26.

Fasted blood glucose (4 hours without food) was performed with standard glucose test strips. Blood was obtained by a tail prick on day 27 of the experiment. Semaglutide affects blood glucose levels while reutericyclin does not.

For each bar graph, one dot represents one mouse in each experimental cohort. Standard error is shown.

For each 2-way ANOVA statistical analyses, injection refers to semaglutide, gavage refers to reutericyclin, and interaction refers to the combination of injection and gavage. No combination effects were noted, indicating that each drug affects the host through distinct mechanisms for the parameters assessed here.

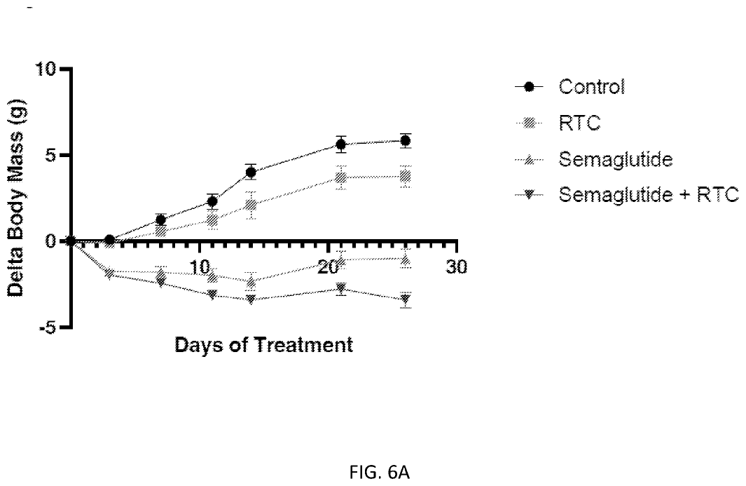

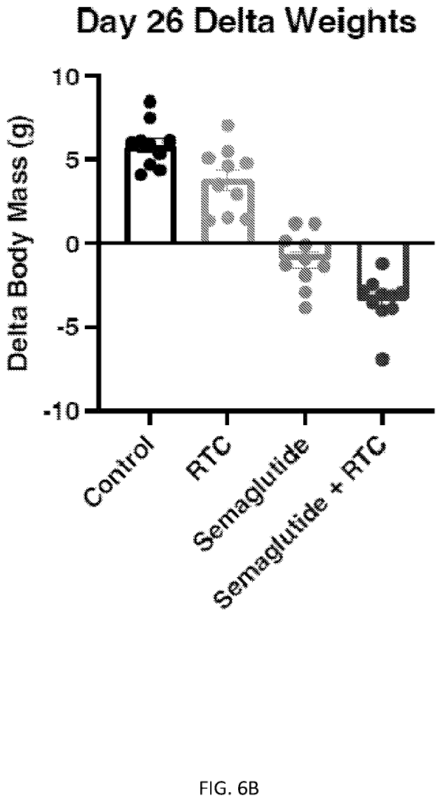

FIGS. 6A and 6B depict two graphs illustrating the weight change of the mice in Example 2. FIG. 6A depicts the change in body mass (grams) over 26 days. FIG. 6B depicts the change in body mass on day 26, with day 1 being the start of the experiment. Table 5 below highlights the 2-way ANOVA analysis of day 26 data from FIG. 6B.

TABLE 5
2-Way ANOVA analysis of Day 26 data

| Source of | % of total | | P value | |
|---|---|---|---|---|
| Variation | variation | P value | summary | Significant? |
| Interaction | 0.04208 | 0.7518 | ns | No |
| Gavage | 7.893 | 0.0001 | *** | Yes |
| Injection | 77.15 | <0.0001 | **** | Yes |

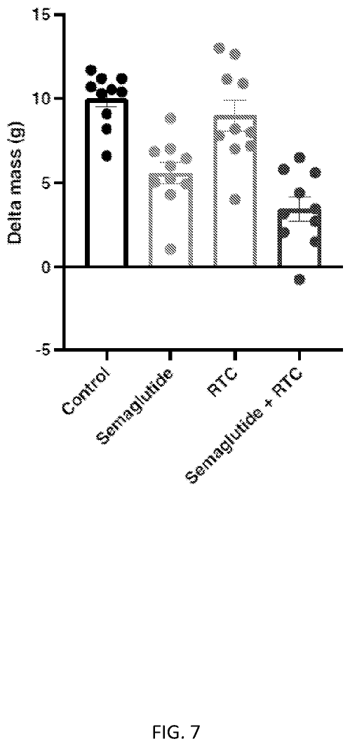

FIG. 7 is a bar graph depicting the change in weight after withdrawal of the semaglutide at day 46 of the experiment described in Example 2. Table 6 below highlights the 2-way ANOVA analysis of day 46 data.

TABLE 6
2-Way ANOVA analysis of Day 46 data

| Source of | % of total | | P value | |
|---|---|---|---|---|
| Variation | variation | P value | summary | Significant? |
| Interaction | 0.7015 | 0.4272 | ns | No |
| Gavage | 5.422 | 0.0319 | * | Yes |
| Injection | 54.71 | <0.0001 | **** | Yes |

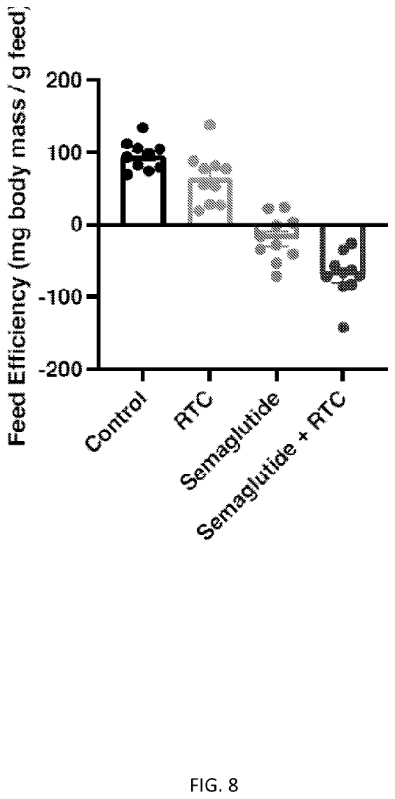

FIG. 8 is a bar graph depicting the feeding efficiency of the mice as described in Example 2. Table 7 below highlights the 2-way ANOVA analysis of this data.

TABLE 7
2-Way ANOVA analysis of feeding efficiency data

| Source of | % of total | | P value | |
|---|---|---|---|---|
| Variation | variation | P value | summary | Significant? |
| Interaction | 0.4610 | 0.3170 | ns | No |
| Gavage | 8.034 | 0.0002 | *** | Yes |
| Injection | 75.39 | <0.0001 | **** | Yes |

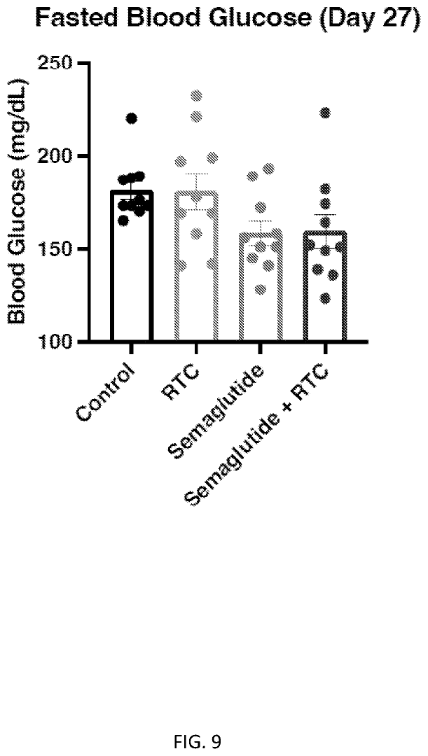

FIG. 9 is a bar graph depicting the fasted blood glucose level at day 27 as described in Example 2. Table 8 below highlights the 2-way ANOVA analysis of this data.

TABLE 8
2-Way ANOVA analysis of the fasted
blood glucose level at day 27

| Source of | % of total | | P value | |
|---|---|---|---|---|
| Variation | variation | P value | summary | Significant? |
| Interaction | 0.02671 | 0.9143 | ns | No |
| Gavage | 9.241e−005 | 0.9949 | ns | No |
| Injection | 18.13 | 0.0077 | ** | Yes |

### NUMBERED CLAUSES

The disclosure herein may also be described in the following numbered clauses.

Clause 1. A method for weight loss in a subject, comprising: administering a therapeutically effective amount of reutericyclin and a glucagon-like-protein-1 (GLP-1) receptor agonist to the subject.

Clause 2. The method of clause 1, wherein the GLP-1 receptor agonist comprises one or more of semaglutide, dulaglutide, exenatide, liraglutide, or lixisenatide.

Clause 3. The method of clause 1 or 2, wherein the GLP-1 receptor agonist comprises semaglutide.

Clause 4. The method of any of clauses 1-3, wherein the reutericyclin and GLP-1 receptor agonist are administered together.

Clause 5. The method of any of clauses 1-3, wherein the reutericyclin and GLP-1 receptor agonist are administered separately.

Clause 6. The method of any of clauses 1-5, wherein the reutericyclin is administered orally.

Clause 7. The method of any of clauses 1-6, wherein the GLP-1 receptor agonist is administered via injection.

Clause 8. The method of clause 7, wherein the injection is a subcutaneous injection or an intramuscular injection.

Clause 9. The method of any of clauses 3-8, wherein the semaglutide is administered in an amount of about 1 nanomole/kg of body weight or less.

Clause 10. The method of any of clauses 1-9, wherein the reutericyclin is administered in an amount of about 100 μg/kg of body weight.

Clause 11. The method of any of clauses 1-10, wherein the administering comprises administering reutericyclin and the GLP-1 receptor agonist on a daily or weekly basis for a first time period.

Clause 12. The method of clause 11, further comprising: subsequent to the first time period, administering reutericyclin to the subject for a second time period, wherein during the second time period the GLP-1 receptor agonist is not administered to the subject.

Clause 13. The method of clause 11 or 12, wherein the administering reutericyclin and the GLP-1 receptor agonist on a daily or weekly basis for a first time period comprises administering reutericyclin on a daily basis and administering the GLP-1 receptor agonist on a daily or weekly basis.

Clause 14. The method of any of clauses 11-13, wherein the first time period is from one day to three years.

Clause 15. The method of clause 12, wherein during the second time period the subject has gained less weight as compared to a subject receiving reutericyclin alone or semaglutide alone during the first time period and receiving reutericyclin alone during the second time period.

Clause 16. The method of any of clauses 11-15, wherein during the first time period when the subject is administered reutericyclin and the GLP-1 receptor agonist, the subject exhibits greater weight loss or weight loss that is substantially similar compared to weight loss exhibited from another subject receiving a dose of the GLP-1 receptor agonist that is at least ten times higher than the dose of the GLP-1 receptor agonist administered to the subject.

Clause 17. The method of any of clauses 1-16, wherein the subject is obese.

Clause 18. The method of any of clauses 1-17, wherein the subject has diabetes.

Clause 19. A method for weight loss in a subject, comprising: administering a therapeutically effective amount of reutericyclin and a sodium glucose cotransporter 2 (SGLT-2) inhibitor to the subject.

Clause 20. The method of clause 19, wherein the sodium glucose cotransporter 2 (SGLT-2) inhibitor comprises one or more of bexagliflozin, canagliflozin, dapagliflozin, empagliflozin, ertugliflozin, ipragliflozin, luseogliflozin, remogliflozin, sergliflozin, sotagliflozin, or tofagliflozin.

Clause 21. The method of clause 19 or 20, wherein the reutericyclin is administered in an amount of about 100 μg/kg of body weight.

Clause 22. The method of any of clauses 19-21, wherein the subject is obese.

Clause 23. The method of any of clauses 19-22, wherein the subject has diabetes.

Clause 24. The method of any of clauses 19-23, wherein the reutericyclin is administered orally.

Clause 25. The method of any of clauses 1-18, wherein the GLP-1 receptor agonist is part of a combination therapeutic.

Clause 26. The method of clause 25, wherein the combination therapeutic comprises the GLP-1 receptor agonist and a GIP (glucose-dependent insulinotropic polypeptide) receptor agonist.

Clause 27. The method of clause 26, wherein the combination therapeutic is tirzepatide.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for weight loss in a subject, comprising:
administering a therapeutically effective amount of reutericyclin and a glucagon-like-protein-1 (GLP-1) receptor agonist to the subject.

2. The method of claim 1, wherein the GLP-1 receptor agonist comprises one or more of semaglutide, dulaglutide, exenatide, liraglutide, or lixisenatide.

3. (canceled)

4. The method of claim 1, wherein the reutericyclin and GLP-1 receptor agonist are administered together.

5. The method of claim 1, wherein the reutericyclin and GLP-1 receptor agonist are administered separately.

6. The method of claim 1, wherein the reutericyclin is administered orally.

7. The method of claim 1, wherein the GLP-1 receptor agonist is administered via injection

8. (canceled)

9. The method of claim 1, wherein the GLP-1 receptor agonist comprises semaglutide and is administered in an amount of about 1 nanomole/kg of body weight or less.

10. The method of claim 1, wherein the reutericyclin is administered in an amount of about 100 μg/kg of body weight.

11. The method of claim 1, wherein the administering comprises administering reutericyclin and the GLP-1 receptor agonist on a daily or weekly basis for a first time period.

12. The method of claim 11, further comprising: subsequent to the first time period, administering reutericyclin to the subject for a second time period, wherein during the second time period the GLP-1 receptor agonist is not administered to the subject.

13. (canceled)

14. (canceled)

15. The method of claim 12, wherein during the second time period the subject has gained less weight as compared to a subject receiving reutericyclin alone or semaglutide alone during the first time period and receiving reutericyclin alone during the second time period.

16. The method of claim 11, wherein during the first time period when the subject is administered reutericyclin and the GLP-1 receptor agonist, the subject exhibits greater weight loss or weight loss that is substantially similar compared to weight loss exhibited from another subject receiving a dose of the GLP-1 receptor agonist that is at least ten times higher than the dose of the GLP-1 receptor agonist administered to the subject.

17. The method of claim 1, wherein the subject is obese or has diabetes.

18. (canceled)

19. A method for weight loss in a subject, comprising:
administering a therapeutically effective amount of reutericyclin and a sodium glucose cotransporter 2 (SGLT-2) inhibitor to the subject.

20. The method of claim 19, wherein the sodium glucose cotransporter 2 (SGLT-2) inhibitor comprises one or more of bexagliflozin, canagliflozin, dapagliflozin, empagliflozin, ertugliflozin, ipragliflozin, luseogliflozin, remogliflozin, sergliflozin, sotagliflozin, or tofagliflozin.

21. The method of claim 19, wherein the reutericyclin is administered in an amount of about 100 μg/kg of body weight.

22. The method of claim 19, wherein the subject is obese or has diabetes.

23. (canceled)

24. The method of claim 19, wherein the reutericyclin is administered orally.

25. The method of claim 1, wherein the GLP-1 receptor agonist is part of a combination therapeutic,
wherein the combination therapeutic comprises the GLP-1 receptor agonist and a GIP (glucose-dependent insulinotropic polypeptide) receptor agonist.

26. (canceled)

27. The method of claim 25, wherein the combination therapeutic is tirzepatide.
